(19) United States
(12) Patent Application Publication
(10) Pub. No.: US 2024/0285267 A1
(43) Pub. Date: Aug. 29, 2024

(54) MOTORIZED END-CUT BIOPSY DEVICE WITH DISPOSABLE SYRINGE AND REUSABLE HANDPIECE

(71) Applicant: University of Florida Research Foundation, Inc., Gainesville, FL (US)

(72) Inventors: Luis Roy ARAYA, Gainesville, FL (US); Hitomi GREENSLET, Gainesville, FL (US); Zachary TUPPER, Gainesville, FL (US); Wayne BRISBANE, Gainesville, FL (US); Samsun LAMPOTANG, Gainesville, FL (US); David LIZDAS, Gainesville, FL (US)

(21) Appl. No.: 18/573,649

(22) PCT Filed: Jun. 22, 2022
(86) PCT No.: PCT/US2022/073097

§ 371 Date: Dec. 22, 2023

Related U.S. Application Data
(60) Provisional application No. 63/213,529, filed on Jun. 22, 2021.

(51) Int. Cl. A61B 10/02; A61B 90/00

(52) U.S. Cl. CPC A61B 10/0283; A61B 90/06; A61B 2010/0208; A61B 2090/378; A61B 2562/0223

(57) ABSTRACT

Various examples are provided related to biopsy devices, systems and methods of use. In one example, a biopsy device includes a housing configured to receive a syringe and a syringe drive assembly that can engage with a cylinder of the syringe when installed in the housing. The housing can secure a plunger of the syringe in a fixed position within the housing and the syringe drive assembly can linearly advance the cylinder within the housing at a constant linear speed when activated. In another example, a biopsy visualization system includes a biopsy device including a housing configured to receive a syringe therein, the syringe including a sensor in an end of the inner stylette, and a visualized tracking and biopsy system that can track the position of the biopsy device from the sensor and generate a 3D visualization including a location of the biopsy device.

### CROSS REFERENCE TO RELATED APPLICATIONS

This application claims priority to, and the benefit of, co-pending U.S. provisional application entitled “Motorized End-Cut Biopsy Device with Disposable Syringe and Reusable Handpiece” having Ser. No. 63/213,529, filed Jun. 22, 2021, which is hereby incorporated by reference in its entirety.

### BACKGROUND

Open surgical biopsy is typically the standard initial diagnostic method to evaluate suspicious breast changes. However, patients risk severe complications during the diagnostic processes. Compared to surgical biopsy, needle biopsy procedures are considered less invasive. Fine-needle aspiration biopsy (FNAB) and core needle biopsy (CNB) are two of the most common needle biopsy procedures. FNAB is performed using a thin (25-20 gauge) needle attached to a syringe to aspirate the tissue from the target area. CNB uses a relatively large (20-14 gauge) coaxial or triaxial needle that can preserve a sufficient amount of the tissue structure for histological analysis. The small size of the FNAB needle allows that biopsy procedure to be less invasive than CNB.

### SUMMARY

Aspects of the present disclosure are related to biopsy devices, systems and methods of use. In one aspect, among others, a biopsy device comprises a housing configured to receive a syringe therein; and a syringe drive assembly configured to engage with a cylinder of the syringe when installed in the housing. The housing can secure a plunger of the syringe in a fixed position within the housing and the syringe drive assembly linearly advances the cylinder within the housing at a constant linear speed when activated. In one or more aspects, the syringe drive assembly can comprise a motor-driven carriage configured to detachably attach to the cylinder of the syringe. The carriage can be linearly driven by a DC motor through a leadscrew connected to the carriage.

In various aspects, the housing can comprise a base handle comprising the syringe drive assembly; and a top cover providing access for installation and removal of the syringe in the housing. The syringe drive assembly can comprise control circuitry and a power source. The power source can be a battery. The base handle can comprise an activation button or trigger to activate the syringe drive assembly. The activation button or trigger can be configured to toggle between states of the biopsy device. In some aspects, the syringe can comprise an inner stylette with a sharp point on a distal end and fixed to the plunger at a proximal end; and a hollow outer cannula or needle with a sharp edge on a distal end and fixed to the cylinder at a proximal end. The outer cannula and inner stylette can mechanically imprint an orientation of an acquired tissue sample. The biopsy device can comprise at least one Hall effect sensor configured to monitor a stroke of the outer cannula, wherein the stroke can be controlled in response to an indication from the at least one Hall effect sensor. A stroke of the outer cannula can be controlled in response to a monitored stall current. The syringe can be a disposable syringe.

In another aspect, a biopsy visualization system comprises a biopsy device, comprising: a housing configured to receive a syringe therein, the syringe comprising an outer needle and an inner stylette including a sensor in a distal end of the inner stylette; and a syringe drive assembly configured to engage with a cylinder of the syringe when installed in the housing, wherein the housing secures a plunger of the syringe in a fixed position within the housing and the syringe drive assembly linearly advances the cylinder within the housing at a constant linear speed when activated; and a visualized tracking and biopsy system configured to track position of the biopsy device based at least in part upon position of the sensor within a patient and generate a 3D visualization including a location of the biopsy device for rendering. In one or more aspects, the sensor can be a magnetic sensor. The outer needle and inner stylette can be fabricated from a non-magnetic material. The sensor can be coupled to the visualized tracking and biopsy system via a cable extending from the sensor through the inner stylette.

In various aspects, stroke position of the syringe can be monitored and altered by Hall effect sensors or stall current. Orientation can be encoded through the mechanical imprinting of the tissue so that the proximal and distal end of the biopsy sample appear unique grossly and under the microscope. Therefore, the core orientation is maintained regardless of tissue sample processing techniques employed for microscopy. The visualized tracking and biopsy system can be configured to concurrently track a micro-ultrasound (microUS) probe. The 3D visualization can include a location of the microUS probe. In some aspects, the 3D visualization can include guidance for obtaining a sample from a region of interest with the biopsy device. The visualized tracking and biopsy system can record position and orientation of the sample within the region of interest. A sample can be obtained by the outer needle, the sample encoded with an orientation indication.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon dearly illustrating the principles of the present disclosure. Moreover, in the drawings, like reference numerals designate corresponding parts throughout the several views.

FIG. 1 illustrates cross-sections of end-cut and side-notch needles, in accordance with various embodiments of the present disclosure.

FIGS. 2A-2C illustrate the principle of an aspiration-assisted end-cut coaxial needle biopsy system, in accordance with various embodiments of the present disclosure.

FIGS. 3A-3H illustrate an example of a biopsy device, in accordance with various embodiments of the present disclosure.

FIGS. 4A-4C illustrate an example of electronic circuitry of the biopsy device of FIG. 3A-3H or 10A-10G, in accordance with various embodiments of the present disclosure.

FIGS. 5A-5C illustrate an example of a control implementation for the biopsy device of FIG. 3A-3H or 10A-10G, in accordance with various embodiments of the present disclosure.

FIG. 6 includes images of a test bench setup, in accordance with various embodiments of the present disclosure.

FIGS. 7A-7C illustrate a comparison of samples acquired by the biopsy device, in accordance with various embodiments of the present disclosure.

FIGS. 8A-8C illustrate examples of an X-cut biopsy needle and acquired samples, in accordance with various embodiments of the present disclosure.

FIG. 9 illustrates an example of 3D visualization guidance for use with the biopsy device including a sensor, in accordance with various embodiments of the present disclosure.

FIGS. 10A-10G illustrate another example of a biopsy device, in accordance with various embodiments of the present disclosure.

FIGS. 11A and 11B illustrate an example of a sensor in an inner stylette of the biopsy device, in accordance with various embodiments of the present disclosure.

### DETAILED DESCRIPTION

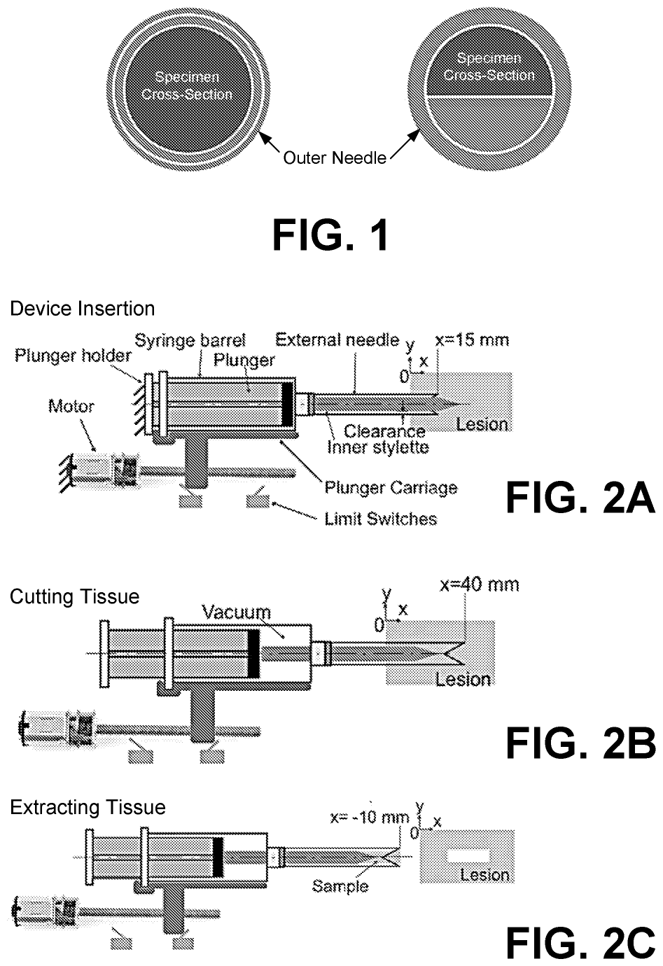

Disclosed herein are various embodiments related to biopsy devices, systems and methods of use. Two types of needles, side-notch needles and end-cut needles are commonly used for core needle biopsy (CNB). Unlike side-notch needles, end-cut needles can collect the full core of the specimen as illustrated in FIG. 1. End-cut needles are normally operated using a spring device; spring-loaded devices tend to push the tissue instead of trapping it. However, regardless of the advantages of the end-cut needles, the high rate of zero biopsy (procedures in which no tissue is collected) has been an issue. A 1995 report stated that the zero-biopsy rate for breast biopsies was reported to be as high as 73%. A constant speed of 1-30 mm/s increases the probability of biopsy success. In addition to reducing capital costs, it is also generally desirable to reduce the need to tether a handheld biopsy device using end-cut needles to sources of mechanical motion, vacuum supply, electrical power, and control. Such tethers may impede positioning of the biopsy device, introduce trip hazards, and increase set-up time. Reference will now be made in detail to the description of the embodiments as illustrated in the drawings, wherein like reference numbers indicate like parts throughout the several views.

According to physicians, a small, easy-to-handle device is desirable—preferably one that is battery operated. A motorized biopsy device and method for obtaining a tissue sample (such as a prostate or breast tissue biopsy sample) are presented. Normally during a biopsy procedure, the entire device is disposed after use; this increases the cost of each procedure. To reduce the cost, the device will be designed such that the disposable parts are minimized, so a standard syringe can be used and fitted inside the designed device, and only the syringe will be discarded after each procedure, and the device can be cleaned and reused. Needle speed also influences the quality of the sample, a range of 1 mm/s to 30 mm/s has been defined to be the most efficient, so an actuator where speed can be controlled inside this range will be chosen.

The biopsy device can include a reusable, disposable syringe with an improved end-cut biopsy needle. The biopsy device can also include a reusable handpiece with a DC motor, controller, and power source to enable convenient and untethered control. The reusable handpiece can incorporate a disposable syringe with an outer cannula or needle attached to the syringe cylinder and an inner solid stylette attached to the syringe plunger. The DC motor can be attached to the cylinder of the syringe with a lead screw, and the motor can move the cylinder of the syringe at a constant speed while also creating a vacuum to trap the tissue sample inside the outer cannula. To remove the trapped tissue, the motor moves backwards, removing the vacuum and expelling the tissue.

The disclosed biopsy device can address these and other problems of the prior art by providing an untethered biopsy device (with a less invasive cannula) that can be inserted into tissue to obtain a full-core biopsy sample by translating a disposable syringe cylinder at a constant speed using a DC motor. The proposed device has the advantage of producing a vacuum in a disposable syringe using a constant-speed DC motor that also translates the outer cannula to obtain tissue samples. The speed of the outer cannula is adjustable, and the stroke of the outer cannula can be adjusted by a Hall effect sensor or stall current. For example, the biopsy device handpiece can have a motorized translation-drive mechanism that engages and drives an external cylinder of a disposable syringe with end-cut needles.

FIGS. 2A-2C illustrate the principle of an aspiration-assisted end-cut coaxial needle biopsy system. The biopsy procedure comprises four elements: insertion, cutting, holding, and extraction. As shown in FIG. 2A, the coaxial needle assembly (including an outer needle) is first inserted to a target position in the suspicious area (e.g., a lesion). From the initial position (e.g., x=0), the motor can advance the inner stylette and needle together. This simultaneous movement can provide superior cutting characteristics because the needle will already be travelling at speed when it begins cutting. After reaching the target position (e.g., x=15 mm), the cutting can be implemented as shown in FIG. 2B. The internal stylette is halted and remains stationary while only the external needle is moved forward (e.g., to x=40 mm) at a constant speed, cutting through the tissue with its tip and storing it. During the cutting process, a vacuum will be created in the syringe body, providing the aspiration that will keep the tissue sample in the needle. This vacuum assists in holding the extracted sample inside the external needle. As soon as the external needle stops, the device is ready to be extracted and the sample recovered as shown in FIG. 2C.

Biopsy devices are often operated in tandem with an ultrasound imaging system so that the clinician can accurately position the needle at the area of concern. Ultrasound probes demand the use of one of the clinician's hands, leaving only one hand for operating the biopsy device. It is thus beneficial for the device design to be able to be operated using one hand only. This would constrain the device's weight, size, and controls.

To be held comfortably during operation without excessive fatigue to the clinician, the biopsy device should be light and securely gripped. For example, a maximum weight of 350 g was determined to be beneficial based on clinician input and existing commercial solutions. Size constraints are often developed by ensuring the device can be grasped comfortably by a hand in the 5^{th} percentile of women. This corresponds to a hand length (measured from the crease of the wrist to the tip of the middle finger with the hand held straight and stiff) of 16.0 cm. It is recommended that the user be able to wrap their hand 270° around the device's circumference. The maximum device circumference was therefore limited to 20 cm. Additionally, the controls need to be easily operated with only one hand without requiring the clinician to change their grip for use during procedures.

The end-cut aspiration-assisted needle biopsy procedure can be performed at insertion speeds between 1 mm/s and 30 mm/s. Higher insertion speeds of 30 mm/s have been shown to improve sample quality in chicken tissue phantoms. For example, to keep motor size and power requirements down, the biopsy device can operate at approximately 1.5 mm/s. This speed corresponds to a motor speed of 180 rpm when used with a M3×0.5 mm lead screw. It was also determined that a 1.08 mm inner stylette with an 18-GA (Ø1.14 mm I.D.) needle can generate an optimal clearance for aspiration assistance and that a 20 deg bevel two-edge geometry needle tip is effective at cutting tissue. While the prototype biopsy device will utilize these features parameters, other speeds, dimensions and geometries may be used.

In addition, it was determined experimentally that a driving force of approximately 25 N was needed to move the syringe forward during the procedure. The device's motor and lead screw can be sized to generate this amount of force. The power screw torque equation was used to determine the maximum torque needed for an M3×0.5 lead screw. The torque equation is given by:

$$T = \frac{F {d}_{m}}{2} ( \frac{L + πμ {d}_{m} sec ( α )}{π {d}_{m} - μ Lsec ( α )} ) ,$$

where T is the shaft torque, F is the force to move the syringe (25 N), dₘ is the difference between the thread's major diameter and half the pitch (2.75 mm), μ is the coefficient of friction between the steel thread and nut (0.14), α is the lead angle (0.053 rad), and L is the thread lead (0.5 mm). Using these values determines a minimum motor torque of 0.68 N-cm or 0.0696 kg-cm. A summary of design features used for the fabrication of a prototype biopsy device is provided in Table 1 below.

TABLE 1

| | | |
|---|---|---|
| Maximum weight | 350 | g |
| Maximum device circumference | 20 | cm |
| Travel speed | 1.5 | mm/s |
| Inner stylet diameter | 1.08 | mm |
| Needle Gauge | 18 | GA |
| Minimum motor torque | 0.0696 | kg-cm |

### Biopsy Device Design

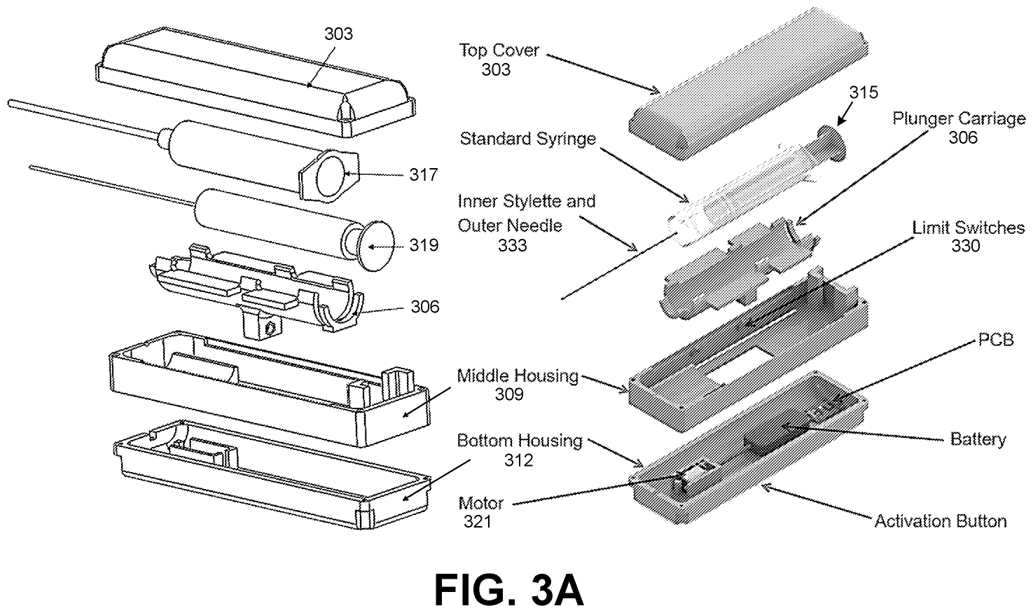

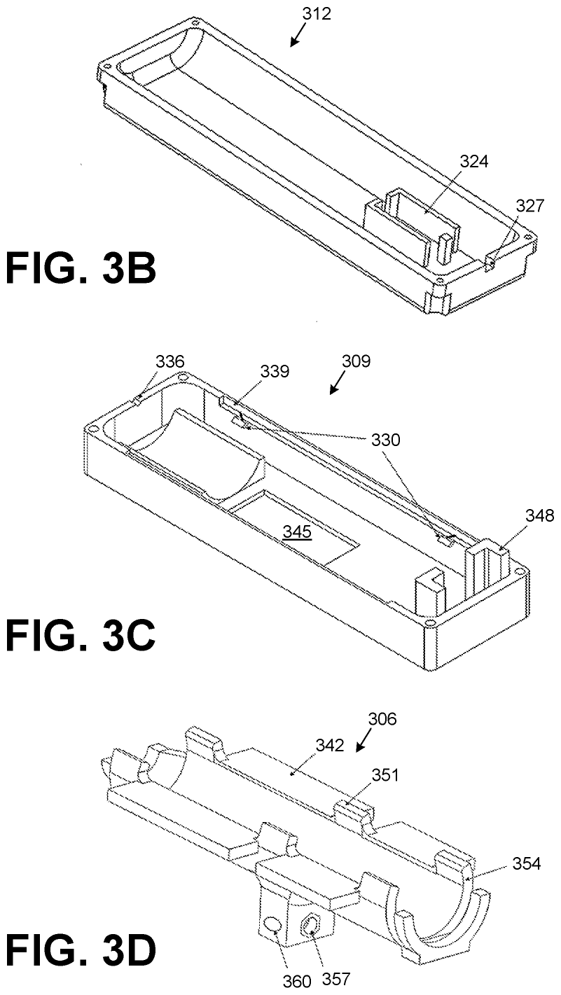

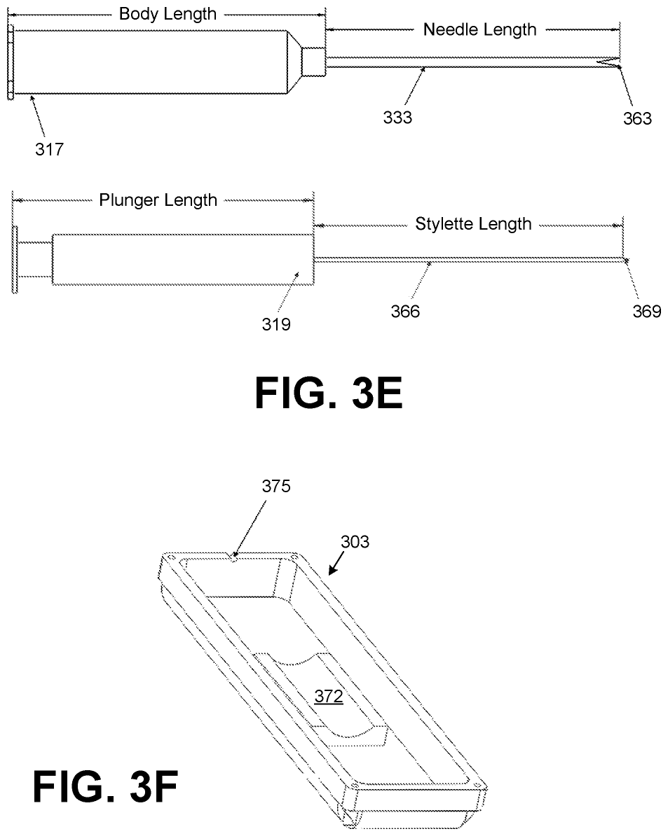

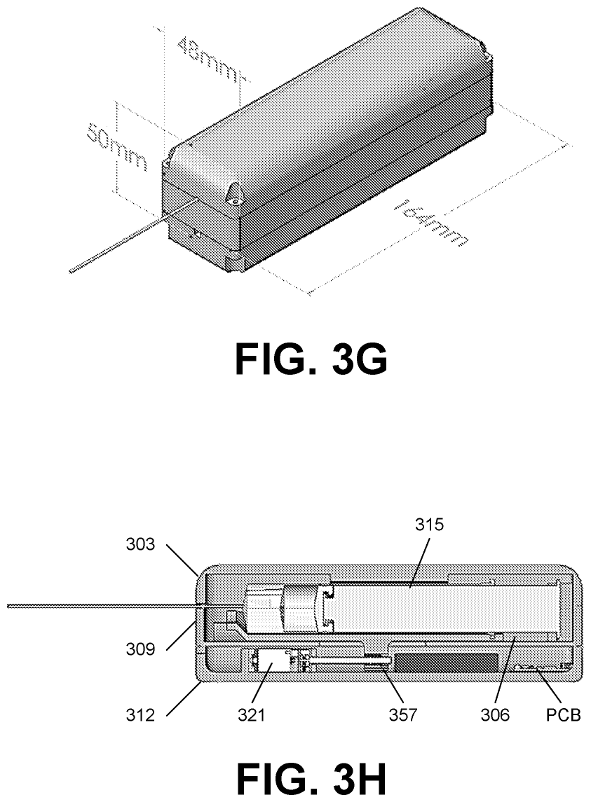

FIG. 3A includes exploded views illustrating examples of components of a biopsy device. As shown, mechanical components of the device body can include a top housing (or cover) 303, a syringe holder (or plunger carriage) 306, a middle housing 309 and a bottom housing 312. The mechanical components mate together and can be joined by fasteners (e.g., M3 screws) at the corners. The biopsy device also includes a syringe assembly 315 comprising a syringe body 317 and a syringe plunger 319.

FIG. 3B shows an enlarged view of the bottom housing 312. The motor 321 (FIG. 3A) and wires can be contained in the bottom housing 312. The bottom housing 312 can include a motor housing 324, which can contain, e.g., a Pololu Micro Gearmotor (rated for a stall torque of 2.4 kg-cm), which meets or exceeds the minimum motor torque as determined above. The motor's output is provided by a lead screw (e.g., a 55 mm long shaft with M3×0.5 threads). The motor receives its power from motor driver (e.g., a DRV8833 motor driver) which is controlled by processing circuitry comprising microcontroller (e.g., an Adafruit Trinket-MO microcontroller). To ensure proper voltage and current are supplied to the motor, an appropriately power supply (e.g., a Kikusui PCR 500LA power supply) can be used to power the motor driver. The motor's wires can be fed through a wire management hole 327 in the bottom housing 312. The wire management hole can also allow for limit switch wires to be fed through.

FIG. 3C shows an enlarged view of the middle housing 309. The middle housing 309 houses the syringe assembly 315, syringe holder 306, and limit switches 330 (FIG. 3A). When positioned in the middle housing 309, an outer needle 333 (FIG. 3A) of the syringe assembly 315 can pass through a hole (or opening) 336 in one end of the middle housing 309. Guide tracks 339 along the sides of the middle housing 309 can constrain the translation of syringe holder flanges 342 (FIG. 3D) in only one direction. This ensures that motion of the syringe assembly 315 is precisely controlled and that the lead screw only bears axial loads with no moments or transverse loads. The limit switches 330 engage with the syringe holder flanges 342 to signal the microcontroller when the syringe has reached the extremes (e.g., beginning and end) of its stroke. For example, the limit switches 330 can be positioned along a guide track 339 such that the syringe holder translates 15 mm between switches. A portion of the syringe holder 306 extends through a slot 345 in the middle housing 309 into the bottom housing 312 to mate with the lead screw. The middle housing 309 can include a plunger stopper 348 that can allow the syringe plunger 319 (and therefore the inner stylette) to advance (e.g., 5 mm) before halting its motion while the syringe body 317 moves forward. This creates the vacuum inside the syringe.

FIG. 3D shows an enlarged view of the syringe holder 306. The syringe holder 306 comprises a carriage to hold the syringe body 317 within the biopsy device. The syringe holder 306 includes an extension configured to mate with the lead screw of the motor 321 and syringe holder flanges 342 configured to engage with the guide tracks 339 (FIG. 3C) of the middle housing 309. Syringe holder flanges 342 mate with corresponding guide tracks 339 to constrain movement of the syringe holder 306 in a linear direction. During operation, the syringe holder flanges 342 translate along the guide tracks 339 and actuate the limit switches 330 (FIG. 3C) as they pass over them. The syringe holder 306 includes two or more syringe holder grips 351, which grip the syringe in a snap fit in the carriage. The syringe can be inserted and removed without tools. A syringe flange slot 354 at one end of the syringe holder 306 can mate with flanges (or tabs) of the syringe body 317 to thoroughly limit its translation in the axial direction. A lead screw nut 357 located within the extension of the syringe holder 306 that passes through the slot 345 of the middle housing 309 and mates with the lead screw of the motor 321. A set screw hole can be included to allow a set screw to be threaded through the extension and contact with the lead screw nut to ensure it remains fixed within the syringe holder 306.

FIG. 3E shows side views of the syringe body 317 and a syringe plunger 319 of the syringe assembly 315. The syringe body 317 can be, e.g., a standard 20 mL syringe with flanges (or tabs) at a proximal end and a Luer lock fitting at a distal end. An outer needle 333 (e.g., an 18 Ga 316 stainless steel needle) can be fitted to the syringe body 317 with a threaded Luer lock. The free end of the outer needle 333 includes a cutting tip 363 (e.g., a specially ground 20 deg cutting tip). The syringe plunger 319 can be, e.g., a standard syringe plunger including a handle at a proximal end and modified to have an inner stylette 366 (e.g., a piece of 304 stainless steel having a diameter of about 1.08 mm) rigidly mounted to a distal end. When the syringe plunger 319 and syringe body 317 are assembled together, the inner stylette 366 nests coaxially within the outer needle 333 with a clearance (e.g., 0.06 mm). The free end of the stylette 366 includes a sharpened tip 369 (e.g., a specially ground sharpened tip). When inserted into the syringe holder 306, the syringe holder grips 351 can engage with an outer surface of the syringe body 317 and the syringe flange slot 354 can mate with flanges (or tabs) at the proximal end of the syringe body 317. When the syringe holder 306 is positioned in the middle housing 309, the handle of the syringe plunger 319 engages with the plunger stopper 348 of the middle housing 309.

FIG. 3F shows an enlarged view of the top housing 303. A syringe guide 372 can ensconce the syringe body 317 to limit its rotation. The outer needle 333 (FIG. 3A) of the syringe assembly 315 can pass through a hole (or opening) 375 in one end of the top housing 303 that corresponds with the hole (or opening) 336 in the middle housing 309.

FIGS. 3G and 3H shows assembled and cross-sectional views of the biopsy device. The overall dimensions of the prototype biopsy device were 50×48×164 mm as shown in FIG. 3G. The overall shape is square on this first prototype to get consistent prints out of the 3D printer used for fabrication of the parts. In the cross section shown in FIG. 3H, the interaction between the motor 321 and the syringe assembly 315 through the syringe holder (or plunger carriage) 306 can be observed. The nut 357 inside the extension of the syringe holder 306 allows for the conversion of rotary motion to linear motion.

### Electronic Design

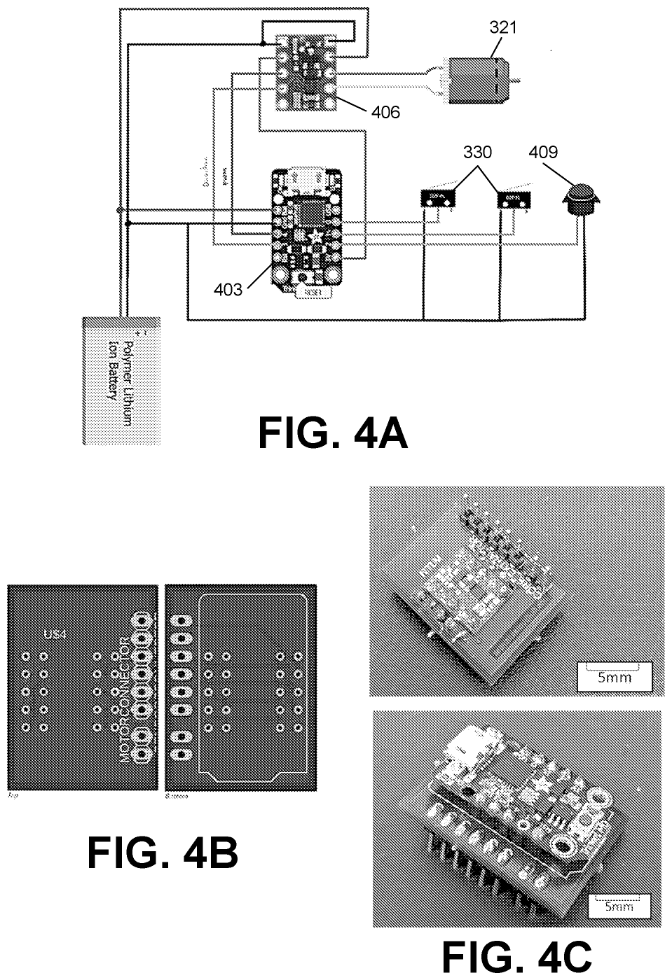

Referring to FIG. 4A, shown is a wiring diagram illustrating an example of the circuitry used to control operation of the biopsy device. The electrical system comprises a microcontroller 403 (e.g., Adafruit Trinket-MO micro controller), a power supply, a motor driver board 406, a micro gearmotor 321 (e.g., a Pololu Micro Gearmotor), two limit switches 330 (e.g., ESE22 limit switches), and a button module 409 (e.g., DAOKI DR-US-162 button module). A small microcontroller 403 (e.g., an ATSAMD21E18 microcontroller) can be used with the motor driver 406 (e.g., a DRV8836 motor driver). As shown in FIG. 4A, a battery can provide power for operation of the motor 321 via the motor driver 406. The limit switches 330 and input button 409 are connected to the microcontroller 403 and used to control operation of the motor 321.

Breakout boards were used for the first prototype and a small PCB was designed to integrate both boards and reduce the use of wires inside the biopsy device. The wiring diagram of FIG. 4A was replicated and designed to fit in a small PCB of 45 mm×20 mm, which is shown in the image of FIG. 4B. The components were assembled and soldered into the PCB. FIG. 12 includes images showing the final PCB with the motor driver 406 and the microcontroller 403 on opposite sides.

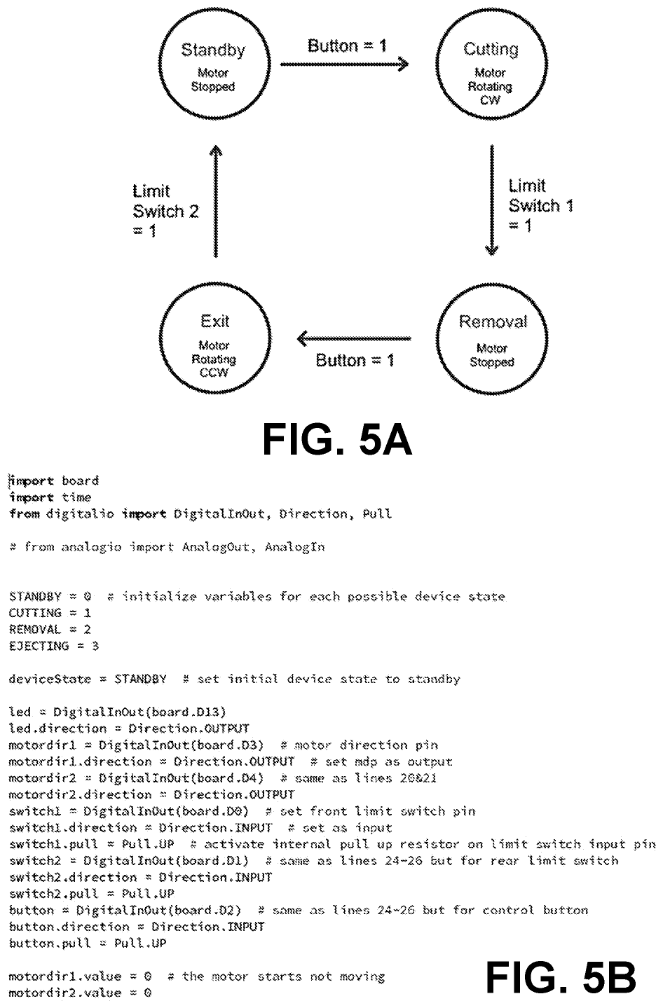

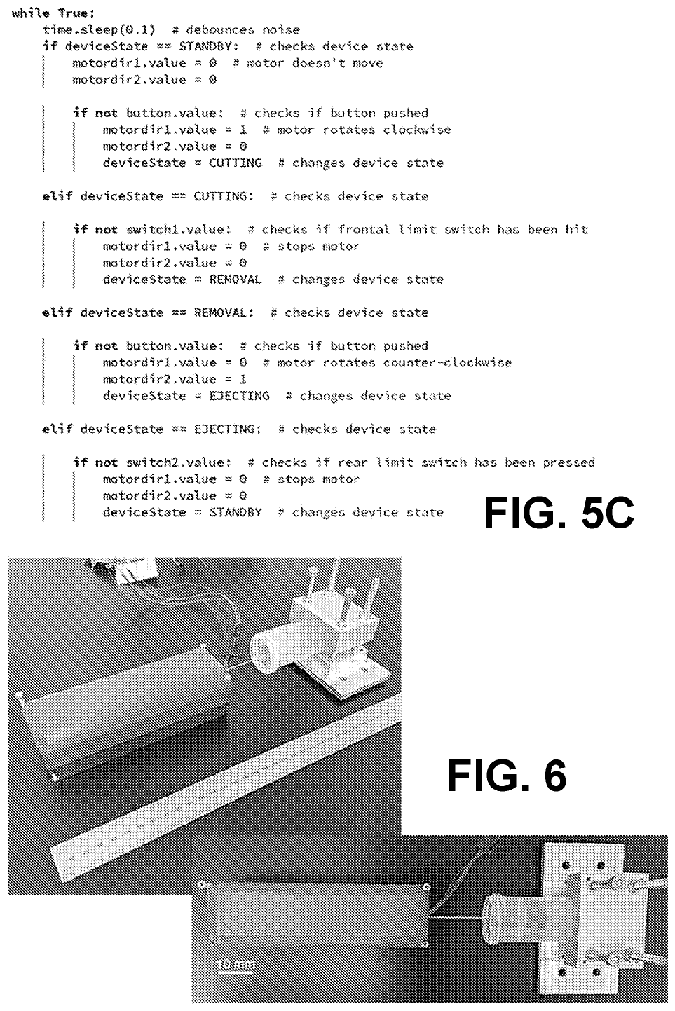

Four states were defined for the device usage: Standby, Cutting, Removal, and Exiting. FIG. 5A illustrates a state machine diagram illustrating operation of the biopsy device between the four states. The device will switch between these states when the activation button is pressed or when a limit switch is reached. STANDBY and REMOVAL states will have the motor 321 stopped, the other two states (CUTTING and EXIT) will rotate the motor 321 clockwise and counterclockwise as indicated. The code running on the microcontroller 403 was developed in python and controls the interactions between all the components.

FIG. 5B shows an example of a setup portion of the code that runs on the microcontroller 403. In the code, the four device states are defined as STANDBY, CUTTING, REMOVAL and EJECTING (or EXIT). When in the STANDBY state, the biopsy device is awaiting input and the motor 321 does not rotate. In the CUTTING state the motor 321 is on and advancing the outer needle 333. In the REMOVAL state the motor 321 is off and the clinician removes the needle from the patient manually. In the EJECTING (or EXIT) state the motor 321 rotates and pushes the outer needle 333 backward, ejecting the sample.

The pins connected to the motor driver board 406 are initialized as outputs. Sending a signal to the IN1 pin of the motor driver 406 causes the motor 321 to rotate in one direction and applying a voltage to the IN2 pin causes the motor 321 to rotate in the other direction. The pins connected to the limit switches 330 and button 409 are initialized as inputs. Internal pull up resistors are activated on these pins to ensure consistent signal. The motor direction pins are both initialized at zero to ensure the motor 321 does not rotate up on startup.

FIG. 5C shows an example of a portion of the code that runs continuously when the biopsy device is powered on. The biopsy device starts in the STANDBY state and changes to the CUTTING state when the clinician pushes the button 409. The motor 321 rotates and the syringe holder 306 moves forward until it activates a frontal limit switch 330, which stops the motor 321 and sets the biopsy device state to REMOVAL. The clinician retracts the biopsy device from the patient's tissue and pushes the button 409 to eject the sample. When the button 409 is pushed, the motor 321 retracts the syringe body 317, causing the inner stylette 366 to move forward in the outer needle 333 and ejects the sample. The motor 321 stops and the state of the biopsy device is reset to STANDBY when the syringe holder 306 hits the rear limit switch 330.

### Design Analysis

Overall Dimensions and Ergonomics. Correct device dimensions are important for preventing upper-extremity musculoskeletal problems. One important feature is the handle. Many studies have researched the topic of tool-handle design to define the optimal size and shape of a tool handle. Most of the studies focus on cylindrical or elliptical shapes. A handle diameter should be in the range of 25 mm to 50 mm according to some studies. If an elliptical shape is chosen, the width to length ratio should be of 1:1.25. As shown in FIG. 3G, the shape of the handle is square on the first prototype to facilitate the 3D printing process. The shape can be changed to an oval that encompasses all the components in a seamless way. The dimensions of the first prototype are on the limit of the optimal size for ergonomics, however this will be improved with the switch to an oval shape.

Inside Layout. The position of the PCB, motor 321 and battery within the bottom housing 312 are illustrated in FIG. 3A. The PCB was positioned to access a charging port from the outside with a simple USB charger. The battery was also conveniently placed to sit in an empty space between the motor 321 and the PCB.

Battery Life Calculation. According to the motor specifications, the no-load current is about 60 mA and the stall current is 750 mA. The battery should be selected so that no recharging is needed during a full day of work. It was assumed that the biopsy device can be operated about 100 times during a full day of work and that a full cycle of the device activates the motor for around 30 s, which activates the motors operating for about 3000 s or 0.83 h. A general recommendation for brush-DC motor operation is that it is run at 25% or less of the stall current, which means an operational current of around 190 mA.

With this current load and the running motor time, it was estimated that a battery of 156 mAh was required. Given the nature of the DC motor load, high burst currents will take place at the initial motor motion. To account for this, a Li—Po battery with high discharge can be used. Li—Po batteries have high power density of around 7.5 kw/kg, which is beneficial since a lot of power can be contained in a small battery. A Tumigy nano-tech battery was selected with 300 mAh, using a factor of safety of 2.

Motor Torque and Speed Calculation. To keep motor size and power requirements down, the biopsy device can be designed to operate at approximately 1.5 mm/s. A motor speed of 180 rpm is needed to satisfy this if used with a M3×0.5 lead screw. It was also determined that a 1.09 mm inner stylette with an 18-GA (01.14 mm ID) generates an optimal clearance for aspiration assistance. A 20-degree bevel two edge geometry needle tip (363 of FIG. 3E) is effective at cutting tissue. The prototype device utilized these parameters. A driving force of approximately 25 N is needed to move the syringe forward during the procedure. The biopsy device's motor 321 and lead screw was chosen to generate this amount of force. As previously discussed, a power screw torque equation was used to determine the maximum torque requirement for an M3×0.5 lead screw

$$T = \frac{{Fd}_{m}}{2} ( \frac{πμ {d}_{m} + L β}{π {d}_{m} β - μ L} ) ,$$

where T is the shaft torque, F is the force to move the syringe (25 N), dₘ is the difference between the thread's major diameter and half the pitch (2.75 mm), μ is the coefficient of friction between the steel thread and nut (0.20), and L is the thread lead (0.5 mm). Inputting these values determines a minimum motor torque of 0.049 kg-cm, which is satisfied by the stall torque of 2.4 kg-cm of the chosen motor.

### Device Testing

Preparation of Gelatin Tissue Phantom. The biopsy device's effectiveness was evaluated based on its performance taking samples from a gelatin tissue phantom. To prepare the gelatin tissue phantom, 7 g of gelatin powder was sprinkled evenly in 50 mL of water and allowed to sit for 5 minutes. This mixture and a separate 50 mL of water were microwaved for 30 s and then mixed and stirred for 2 minutes. The mixture was poured into a cylindrical container and kept at room temperature for one hour, then covered and transferred to a refrigerator for 24 h. Test samples were removed from the refrigerator and allowed to sit for 1 hour prior to testing.

Preparation of Needle and Inner Stylette. 18GA (1.27 mm OD, 1.14 mm ID) 316 stainless steel capillary tubing was used to form the outer needle 333. The tubing was cut to a length of 100 mm and one end was shaped with a 20-degree bevel two-edge geometry. The outer needle 333 was inserted into a Luer-lock fitting, hot-glued in place, and mated with a 25 mL syringe body 317. The inner stylette 366 was made from a 304 stainless steel rod (Ø1.08×127 mm) and a 20-degree bevel was added to its tip. This inner stylette 366 was rigidly attached to the syringe plunger 319 such that it protruded from the plunger's tip and their central axes were colinear.

Experimental Setup Test Bench. FIG. 6 includes images showing isometric and top view of the experimental setup. The gelatin tissue phantom was put into a fixture and taped to the test bench using double sided tape. The syringe body 317 and syringe plunger 319 were assembled and inserted into the syringe holder 306. It was manually verified that the syringe plunger handle was in contact with the rear wall of the middle housing. The biopsy device was closed and taped to the test bench such that the needle tip was perpendicular to the gelatin surface and just barely touching it. The motor driver board was hooked up to the power supply and supplied with 7.4 V, 5 V, or 3.3 V.

The button was pushed, and the biopsy device was allowed to cycle on its own. Once the biopsy device had entered the REMOVAL state, the tape attaching it to the test bench was removed and the biopsy device was pulled back manually at a speed of approximately 1.5 mm/s. Emphasis was placed on limiting the device's retraction to steady translation in only one direction with minimal side-to-side oscillation.

A petri dish was placed below the needle tip and the button 409 was pushed again, setting the device to the EJECTING state. The outer needle 333 and inner stylette 366 were allowed to fully retract, pushing out the sample. The sample was placed in a petri dish. Its length was measured from tip to tip using a ruler with mm demarcations. Its mass was measured using a Mettler Toledo AB265-S/FACT microgram scale with a resolution of 0.1 mg. This process was repeated five times for each voltage.

In addition to testing the biopsy device when it was taped to the test bench, the biopsy device was tested when it was held in the experimenter's hand. This aimed to test the biopsy device as it would be used in a clinical setting. The testing procedures are effectively identical, with the only difference being the way the biopsy device is secured in space. A summary of the experimental conditions is provided in Table 2 below.

TABLE 2

| | |
|---|---|
| Needle | 316 stainless steel (Ø1.27 mm OD × |
| | Ø1.14 mm ID × 100 mm) |
| Needle tip geometry | 20 deg bevel two-edge geometry |
| Inner stylet | 304 stainless steel (Ø1.08 mm × 100 mm) |
| Motor driver board voltage | (a) 3.3 V |
| | (b) 5.0 V |
| | (c) 7.4 V |
| Device positioning | (a) Test bench |
| | (b) Handheld |

Because of insufficient power, the biopsy device was found to be incapable of properly cycling when the motor driver board 406 was supplied with 3.3 V. However, the biopsy device successfully acquired samples when the motor driver board 406 was supplied with 5.0 V and 7.4 V (at speeds of 1.34 mm/s and 1.51 mm/s respectively).

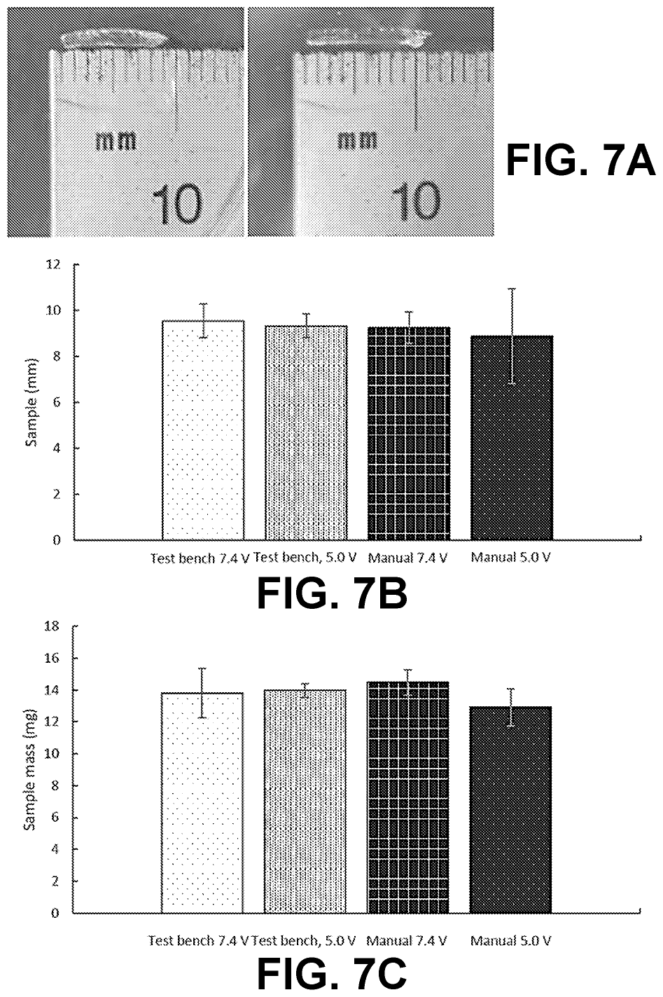

The samples acquired by the biopsy device in all testing arrangements except the 3.3 V arrangement were of near-perfect quality. No sample deformation, stretching, or fracturing was observed, as shown in FIG. 7A. No sample displayed “head structures”, a deformation in which the sample tip is stretched and deformed. No zero biopsies occurred in any of the 20 trials.

FIG. 7B shows the average sample length for each of the testing arrangements. Sample lengths were measured using the image analysis software ImageJ and averaged across 5 trials. There was no significant difference between the 7.4 V and 5.0 V test bench sample lengths, indicating that needle travel speed has no significant effect on sample length. The average test bench sample length (9.43±0.62 mm) and the average manual sample length (9.05±1.07 mm) were not significantly different, indicating the device produces equally long samples independent of fixture method. The average standard deviation of the sample lengths produced in the test bench arrangement (±0.62 mm) was less than that of the average standard deviation of the manual sample lengths (±1.37 mm). This indicates that manually holding the machine produces samples of less consistent length than the test bench fixture method.

FIG. 7C shows the masses of the samples. Sample masses were measured using a microgram scale and averaged across 5 trials. The trends in sample mass are similar to those in sample length. Voltage has no significant effect on average sample mass, nor does fixture method. Fixture method has no significant effect on sample mass variance. However, in contrast to the sample lengths, fixture method does not appear to affect sample mass consistency (both fixture methods produce samples with a mass variance of 0.98 mg).

The tissue phantom samples were observed to have minimal damage, which can produce more reliable results during diagnosis. No zero biopsies were observed in any of the 20 trials conducted, giving the device a 0% zero biopsy rate within this study. By design, the stroke length and therefore the sample length are shorter than that of other trials conducted in this way, but sample lengths and sample masses are proportionally in line with previous findings which yielded samples of 22 mm with a 25 mm cutting length (88% sample/stroke ratio). The average sample/stroke ratio in this study was 92.4%.

### Biopsy System Design

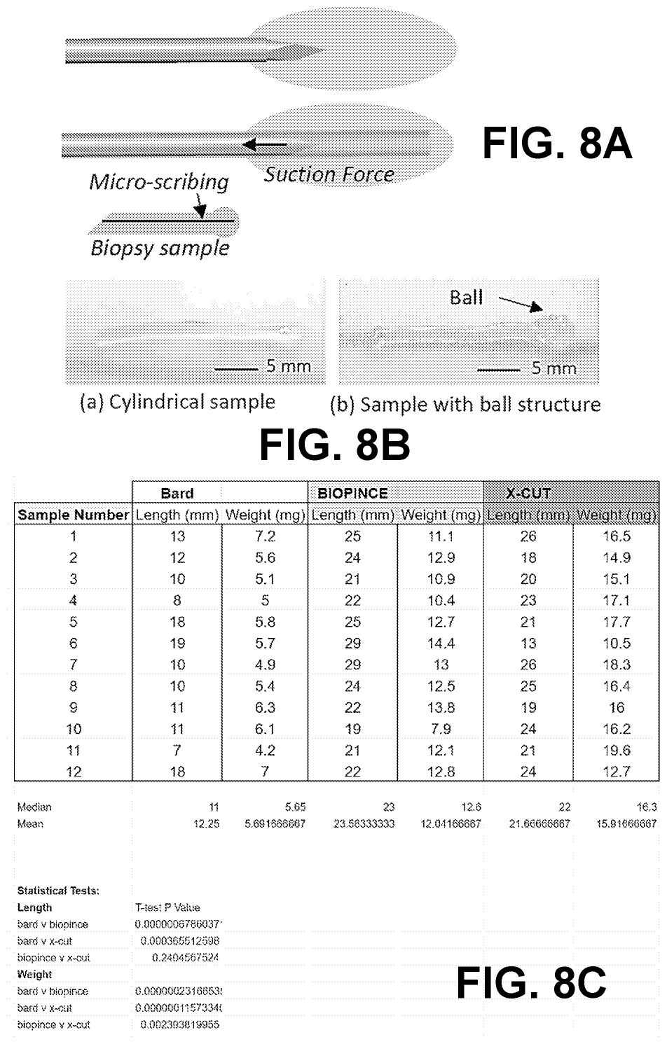

A motorized handheld aspiration-assisted biopsy device, which is used in combination with a Visualized Prostate Biopsy System (vPBx) and micro-ultrasound (microUS), can precisely guide the needle to a desired position, extract a large and dense tissue sample, and encode the biopsy core position and orientation within the prostate on the collected sample. Use of an end-cut (e.g., X-cut) needle in the biopsy device can offer advantages in sample acquisition. FIG. 8A illustrates an example of the X-cut needle-biopsy procedure with functionality to encode position and orientation on samples. On the distal end, the X-cut biopsy sample can have a ball structure that can be used to encode directional information. Ball size can be adjusted by changing the coaxial-needle clearance. FIG. 8B shows representative gelatin samples taken with 18-gauge coaxial needles. The clearance between the outer needle and the internal stylette was 25 μm in the cylindrical sample image (a) and 50 μm in the ball structure sample image (b). The needle-penetration depth was set at 25 mm in the tests. In both cases, the X-cut biopsy sample length was 25 mm, which was 100% of the expected sample. In contrast, existing side-cut and end-cut needles collect 87% and 58-70% of the expected lengths, respectively. A preliminary comparison of the X-cut device compared to the Bard Side-Cut and Argon Biopince biopsy guns in a bovine cardiac muscle model demonstrated a significantly better tissue sample weight for the X-Cut device (p<0.01). FIG. 8C includes a table comparing the three biopsy methods. The core length was comparable between the Biopince and X-cut devices (p=0.24), both of which were significantly longer than the Bard device (p<0.01). The X-cut biopsy concept enables a precision biopsy without unnecessary over-cutting, which is significant. If the rotational information is encoded on the sample and combined with needle-guidance and cancer-imaging systems such as, e.g., vPBx and microUS, the collected sample can inform pathologists and urologists of the sample's position and orientation more completely and more accurately.

A motorized, reusable, aspiration-assisted coaxial end-cut needle-biopsy system using a standard disposable syringe can be operated with a single hand. Encoding of the sample orientations (e.g., tapering and generating a ball structure on one end of the core to differentiate the distal end from the proximal end) can be while generating a large diameter full-core tissue sample (i.e., the sample length is equal to the external-cannula-penetration length). Micro-scribing of the tissue sample may be used to encode rotational information while extruding the sample from the external cannula. Production of a zero-biopsy rate of 0% is possible based upon testing of the biopsy device. Excessive tissue deformation caused by a spring-loaded actuator may be minimized, thus minimizing collateral tissue damage. Reduction of the spring actuation noise can also reduce discomfort during biopsy. This can overcome limitations of the conventional side-cut needle-biopsy devices.

In addition, integration of vPBx tracking with the X-cut biopsy device can guide the needle to the desired location and record the position and orientation of each core. The combination of the X-cut needle, vPBx, and microUS can provide a precision needle-guidance and tracking system that can help identify and record each sample location and orientation for accurate cancer detection and treatment planning. This can eliminate unnecessary damage to the tissue/organs and facilitate patient recovery.

The principle of the aspiration-assisted X-cut coaxial needle biopsy system is similar to the principle illustrated in FIGS. 2A-2C. The biopsy procedure comprises five steps: insertion, cutting, holding, extraction, and extrusion. As shown in FIG. 2A, the coaxial needle assembly is inserted to a desired location. After reaching the target position (e.g., x=15 mm), the outer needle can be moved forward at a constant speed by a stepper motor, cutting through the tissue as shown in FIG. 2B while the internal stylette remains stationary. During the cutting process, a vacuum will be created in the syringe. This vacuum assists in holding the extracted sample inside the outer needle. As soon as the outer needle stops, a block can be placed between the plunger holder and the syringe body to lock the plunger in position (e.g., x=40 mm). The coaxial needle can then be removed while maintaining the block, extracting the sample from the patient as shown in FIG. 2C. Instead of the sampled core being shaken loose and landing as it may (as is current practice), when the outer needle is retracted by reversing the stepper motor, the sampled core is extruded and deposited into a receptacle in a known position and orientation.

Image-Guided Biopsy. Most prostate biopsies are performed using conventional transrectal ultrasound (TRUS), which poorly discerns prostate cancer, and freehand systematic biopsy. This “blind” technique was believed to be inferior to new MRI-based biopsy strategies, in which the tumor is imaged and targeted using MRI/TRUS fusion. However, the MRI/TRUS fusion biopsy still misses up to 25% of clinically significant prostate cancer (csPCa). Additionally, the registration software used to align MRI and ultrasound can systematically introduce errors. To compensate for the accuracy limitations, the biopsy strategy for the last decade has been to perform additional biopsies (i.e., fusion biopsy immediately followed by systematic prostate biopsy (sPBx)). According to a retrospective review of 30,191 biopsies taken at two different institutions using the combined MRI targeted and systematic strategy, only 12% of cores yielded csPCa.

Imaging modality, high-frequency microUS, offers higher resolution (e.g., a 300% increase) and improved soft-tissue contrast compared to conventional TRUS. Multiple studies have demonstrated that microUS is capable of imaging prostate tumors and having accuracy comparable to MRI. Initial evaluation comparing MRI with microUS demonstrated that microUS has a high sensitivity while MRI has a high specificity for cancer-tumor imaging. Combining MRI and microUS imaging modalities enables synergistic imaging strengths. Importantly, imaged with the combined MRI and microUS imaging systems can avoid missing tumors in prostates.

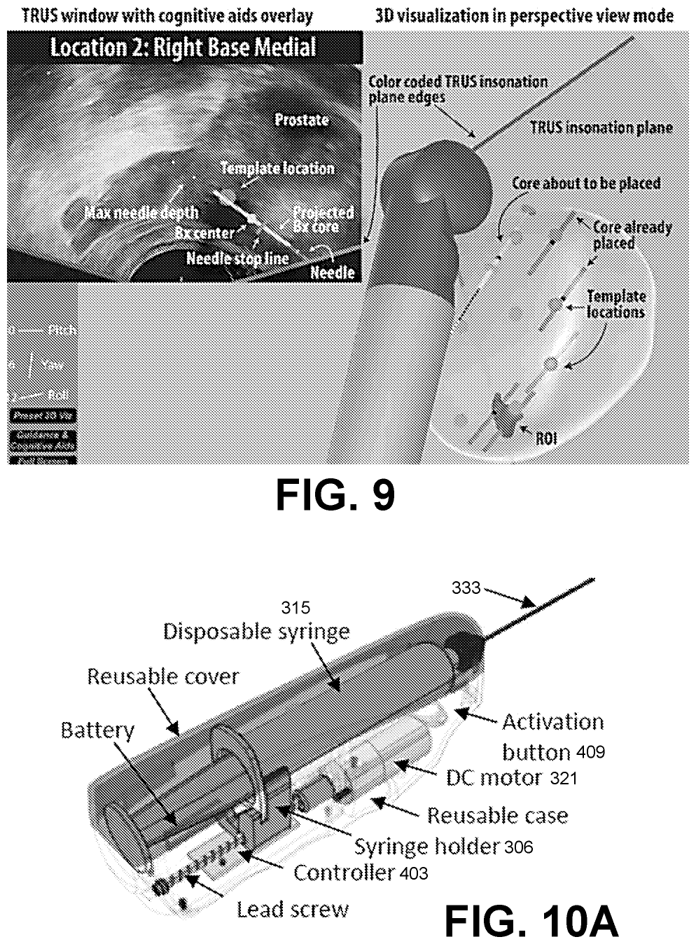

Three-dimensional (3D) Visualized Prostate Biopsy System (vPBx). State-of-the-art PBx guidance systems like the UroNav fuse a prior MRI image with TRUS imaging to provide targeted biopsy of regions of interest (ROIs) identified via MRI. While generally believed to be superior in detecting PCa compared to freehand sPBx using TRUS only, MRI/TRUS fusion biopsy was instead found to be non-inferior in a recent, well-designed, retrospective study with csPCa (Gleason ≥7) detection rate of 17% vs. 18% for freehand sPBx. The improved biopsy-core characteristics of the X-cut biopsy device can be enhanced by integrating it with a vPBx planning, guidance, and feedback system. In an initial implementation, vPBx reduced prostate biopsy false negatives (PBxFNs) from 52% to 2% for 0.5 mL spherical apical lesions without using MRI. Unlike current commercial guidance systems, vPBx can track the biopsy needle and prostate in 6 degrees of freedom (DOF) to 0.2 mm resolution and can provide a real-time 3D visualization of the prostate, TRUS probe, biopsy device, and cognitive aids for planning, guidance, and feedback. FIG. 9 illustrates an example of 3D visualization guidance with a 2D TRUS overlay.

The vPBx can be retrofitted to an ExactVu machine and EV-29L microUS probe via a 3D-printed removable clip that snaps on to the probe and contains a 6 DOF magnetic sensor. The microUS image is transferred in real-time via High-Definition Multimedia Interface (HDMI) to a vPBx computer with touchscreen where the microUS image is replicated without distortion and used by the vPBx software. The prostate can be tracked via a miniature sensor imbedded in a urinary catheter placed in the bladder neck prior to the prostate biopsy procedure and removed as soon as 3D guidance is no longer needed. The X-cut biopsy device will be integrated into vPBx by placing a miniature 6 DOF sensor in the tip of its hollow inner stylette. In the vPBx, cognitive aids can be overlaid on both the 2D TRUS display and the 3D visualization to show the segmented ROI, intended sPBx template location, needle-stop line (which indicates how far to insert the needle tip before moving the outer needle for cutting), yellow line indicating location of the tissue about to be sampled, and red stop line indicating maximum needle tip excursion. The lines can move as the tracked X-cut biopsy needle is moved.

In situ prostate and ROI segmentation can be performed in the urology clinic. The prostate can be roughly segmented by the urologist by manually tracing the contour of the prostate on the replicated 2D microUS image for different prostate slices. This pragmatic segmentation takes 1-2 minutes and results in a 3D prostate displayed in the vPBx graphical user interface. The urethra and any ROI visible in the microUS image can also be segmented. Lesions are more visible in microUS than in traditional TRUS, which creates synergy between vPBx and microUS. For planning, vPBx supports rapid user-adjustment of sPBx-template locations or target locations to the segmented 3D prostate and ROI(s). Users can select different sPBx templates with a different number and pattern of cores that they drag as a 3D set of points into the segmented prostate and adjust, including adding locations.

Preliminary data was obtained from a vPBx with 48 urology residents and faculty members using the vPBx with simulated TRUS imaging for systematic prostate biopsy. Template deviation and PBxFN percentage for a 0.5 mL virtual spherical lesion at the apex during simulated sPBx were reduced from 11.8 mm and 52% with traditional TRUS guidance to 2.5 mm and 2% in the proof-of-concept vPBx. The significant reduction of mean template deviation to 2.5 mm (p<0.0001) and the significant reduction of PBxFN percentage (p<0.001) with the vPBx suggest that PBxFN percentages will also be reduced with the vPBx integrated with actual microUS equipment and the X-cut biopsy device.

### Biopsy Device Design

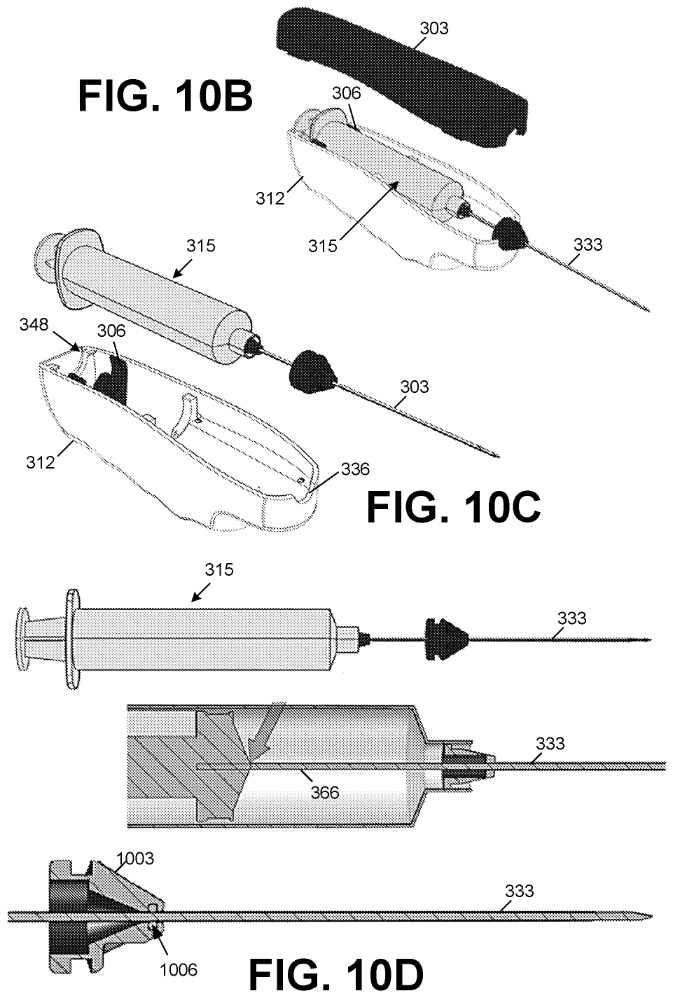

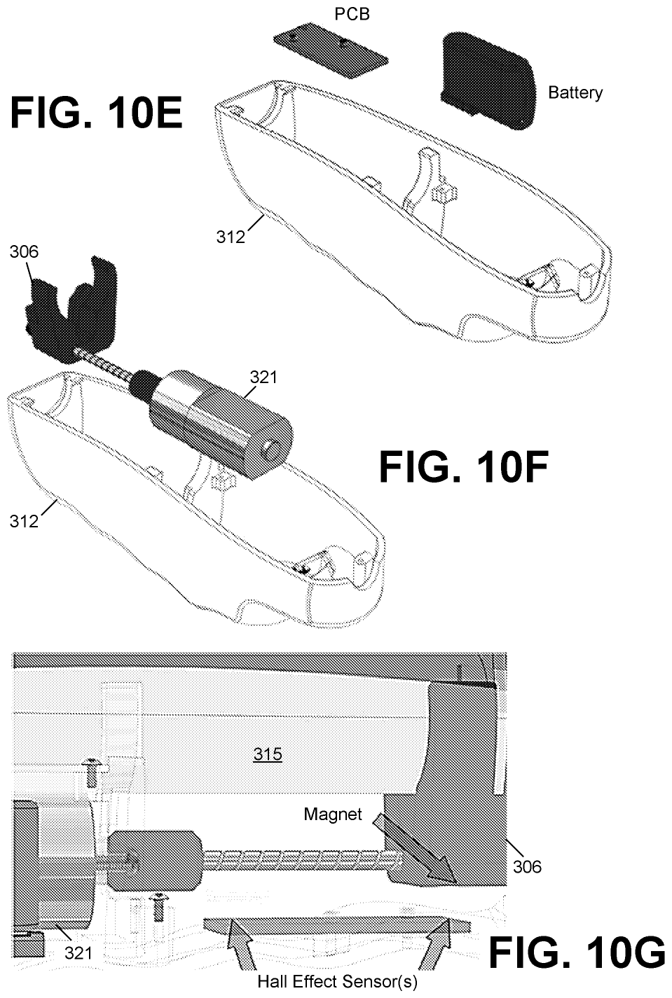

FIGS. 10A-10G illustrate another example of a biopsy device. The biopsy device includes a reusable handpiece including two housing portions: a top cover 303 and a bottom housing or case 312, a disposable syringe 315 with a coaxial end-cut biopsy needle (including an internal stylet 366 and an outer needle 333), a DC motor 321, a controller 403, and a battery as a power supply, all of which combine to enable convenient, one-hand control. FIG. 10A illustrates the components positioned within the housing. The housing has an oval cross-section with an ergonomic design for gripping with a single hand. As illustrated in FIG. 10B, the top cover 303 can be removed for access to an installed syringe assembly 315 for replacement. The syringe assembly 315 is secured within the bottom housing by a syringe holder 306 FIG. 10C illustrates the syringe assembly 315 removed from the syringe holder 306 and bottom housing 312. The syringe assembly 315 can be disposable while the rest of the biopsy device is reusable.

The reusable handpiece incorporates a disposable 20 mL syringe that comprises an outer needle 333 attached to the syringe body 317 and an inner stylette 366 attached to the syringe plunger 319. As shown in FIG. 10D, the outer needle 333 can include a needle holder 1003 to position the outer needle 333 within the hole (or opening) 336 in the bottom housing 312 as illustrated in FIG. 10B. The needle holder 1003 can include a needle O-ring 1006 to align the outer needle within the needle holder 1003. The inner stylette 366 is fixed to the syringe plunger 319 as indicated by the large arrow in FIG. 10D and extend coaxially through the outer needle 333. As shown in FIG. 10E, the PCB including the microcontroller 403 and motor driver 406, and the battery, are positioned within the bottom housing 312. The motor 321 (e.g., a DC stepper motor) is coupled to the syringe holder 306 by a lead screw that can engage with a nut 357 in the syringe holder 306 as shown in FIG. 10F. Limit switches 330 or Hall effect sensors (as indicated by the large arrows in FIG. 10G) can be used to detect movement of the syringe holder 306 and syringe assembly 315. A magnet can be included on a surface of the syringe holder 306 as shown in FIG. 10G to facilitate sensing with the Hall effect sensors.

The coaxial needle can be manually inserted into a patient's prostate, and when the coaxial needle reaches the desired location, the activation button 409 can be depressed to turn on the DC motor 321. The motor 321 moves the syringe body 317 at a constant speed. The outer needle 333 (attached to the syringe body 317) also moves forward at a constant speed to the desired position, cuts tissue, and creates a vacuum to retain the tissue inside the outer needle 333. The speed and stroke (penetration depth) of the outer needle 333 movement are adjustable and programmable as was previously described. The stroke of the outer needle 333 influences the cut length of tissue. Control of these variables can enable the biopsy device to take a tissue sample that is larger and denser than samples taken by existing biopsy devices. After cutting the sample, the coaxial needle can be manually removed from the prostate while holding the tissue sample, and the tissue sample is thus detached from the patient. While holding the tracked X-cut device in a known position and orientation, the button 409 can be depressed a second time to move the syringe body 317 back to its original position and to extrude the full-core tissue sample from the outer needle 333 while also micro-scribing the sample to encode the sample rotation.

The biopsy device will enable one-hand operation for all tasks—from inserting the coaxial needle into the patient body, to cutting and extracting tissue, to extruding the tissue from the outer needle 333. By combining the X-cut device with vPBx and microUS, the coaxial needle can be precisely guided to a template or target location. The X-cut device can be used via transperineal needle guides that attach to the microUS probe, and a 6 DOF magnetic sensor can be embedded in the tip 369 of the inner stylette 366 and communicate needle position and orientation to the vPBx. The sensor integration can be carried out during the design and fabrication of the inner stylette 366.

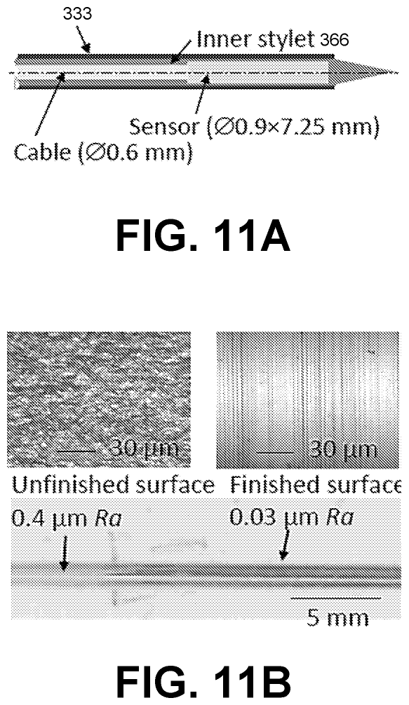

Fabrication of Coaxial Needle with Functionality to Encode Position and Orientation on Samples. FIG. 11A shows an example of the coaxial needle that can be designed and manufactured in this task. The position sensor can be, e.g., 0.9 mm in diameter and 7.25 mm long with an attached cable (e.g., 0.6 mm in diameter, 3.3 m long). To mount this sensor inside the inner stylette 366, the inner stylette 366 can be made from 18-gauge 316L stainless steel (annealed), Inconel tube or other appropriate material. Both materials are non-ferromagnetic. One end of the tube interior can be precisely machined for the sensor to be positioned at the designated location. A cone tip can then be attached to the end of tube. The cable can pass through the other end of the tube (inner stylette 366) and through the syringe plunger 319 for connection to the vPBx system.

A 16-gauge 316L stainless steel (annealed) or Inconel tube can be used for the outer needle 333. The needle-tissue interaction force, which can cause patient pain, decreases with increasing needle-penetration speed and is influenced by the needle surface roughness. In the biopsy device, both the exterior and interior of the outer needle 333 can be in contact with tissue and can be smoothed to reduce the needle-tissue interaction force. The feasibility of Magnetic Abrasive Finishing (MAF) has been demonstrated for internal and external surface finishing of 316L stainless steel capillary tubes. Accordingly, MAF can be used to smooth the outer needle surfaces.

FIG. 11B shows a representative 18-gauge 316L stainless steel needle (unfinished and finished surfaces polished using MAF). The surface textures (e.g., roughness and lay) can be altered by changing the abrasive size and motion. For example, scratch marks can be made in the axial direction (that is, the tissue-sliding direction), which will influence the needle-tissue interaction force. Using MAF, the internal and external surfaces roughness and texture of a 16-gauge external cannula will be altered to various levels (e.g., 0.01-0.1 μm Ra). Note that MAF is applicable for both annealed 316L stainless steel and Inconel. After finishing the outer needle 333, the tip of the outer needle 333 can be shaped for, e.g., X-cut operation.

To record the rotational orientation of the sample, micro-scribing and tapering the sample at a pre-determined position (see FIG. 8A) can be simply implemented during extrusion of the sample from the outer needle 333. The efficacy of encoding the rotational orientation, the geometry and force of the micro-scribing and tapering tool can be determined.

The X-cut biopsy device and the core position and orientation can be tracked in 6 degrees of freedom using, e.g., an NDI Model 90 magnetic sensor embedded in the inner stylette 366. Both the inner stylette 366 and the outer needle 333 of the X-cut biopsy device can be made of Inconel tubing that does not magnetically interfere with tracking by the NDI sensor. The tracking data can be used to update in real-time a 3D visualization of the X-cut biopsy device and cognitive aids associated with the X-cut biopsy device characteristics (e.g., variable core length, desired core position, predicted core position, predicted deviation from desired core position, needle tip stop line, and/or maximum needle tip excursion). The X-cut can continue to be tracked while held in a predetermined orientation (verified by the tracking) relative to a core receptacle when the core is extruded so that the extruded cores remain oriented. The tracking and 3D visualization of the X-cut biopsy device can be performed using, e.g., the SMMARTS SDK.

A microUS probe can be tracked in 6 degrees of freedom using, e.g., a model 800 NDI sensor embedded in a removable clip attached to, e.g., an EV29L microUS probe. Preliminary tests have established that the EV29L microUS probe will not interfere with magnetic tracking by the NDI sensor. A CAD file of the EV29L probe can be used to create a 3D model of the EV29L probe for use in the 3D visualization provided by the vPBx, which can be executed on a computing device including processing circuitry (e.g., processor and memory) such as, e.g., on a laptop computer, tablet or other appropriate processing device. The tracking and 3D visualization of the EV29L probe, including visible insonation planes with color-coded edges and yaw, pitch, and roll indicators can be performed in Unity using the SMMARTS SDK.

The vPBx can be designed to be retrofitted to existing imaging equipment such as the Exact Imaging ExactVu, which may be used here. Accordingly, the ultrasonography image can be transmitted to the vPBx's computer in real time and without distortion so that the replicated image can be used, for example, for in situ segmentation of the prostate and ROIs. Image transmission can be via HDMI or other appropriate communication link. A tracked X-cut biopsy needle and a tracked microUS probe can be integrated into the vPBx guidance system for transperineal systematic and targeted prostate biopsy. The tracking and 3D visualization of the X-cut biopsy device and EV29L microUS probe can be implemented as discussed during a sPBx workflow for transperineal prostate biopsy. This can include creating a feature that allows users to customize a sPBx template to the in situ segmented prostate, moving and adding template locations and setting the sequence in which the template locations will be sampled. For targeted biopsy, in situ segmentation and targeting of ROIs visible in microUS will be built and verified.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A biopsy device, comprising:
a housing configured to receive a syringe therein; and
a syringe drive assembly configured to engage with a cylinder of the syringe when installed in the housing, wherein the housing secures a plunger of the syringe in a fixed position within the housing and the syringe drive assembly linearly advances the cylinder within the housing at a constant linear speed when activated.

2. The biopsy device of claim 1, wherein the syringe drive assembly comprises a motor-driven carriage configured to detachably attach to the cylinder of the syringe.

3. The biopsy device of claim 2, wherein the carriage is linearly driven by a DC motor through a leadscrew connected to the carriage.

4. The biopsy device of claim 1, wherein the housing comprises:
a base handle comprising the syringe drive assembly; and
a top cover providing access for installation and removal of the syringe in the housing.

5. The biopsy device of claim 4, wherein the syringe drive assembly comprises control circuitry and a power source.

6. The biopsy device of claim 5, wherein the power source is a battery.

7. The biopsy device of claim 4, wherein the base handle comprises an activation button or trigger to activate the syringe drive assembly.

8. The biopsy device of claim 7, wherein the activation button or trigger is configured to toggle between states of the biopsy device.

9. The biopsy device of claim 1, wherein the syringe comprises:
an inner stylette with a sharp point on a distal end and fixed to the plunger at a proximal end; and
a hollow outer cannula with a sharp edge on a distal end and fixed to the cylinder at a proximal end.

10. The biopsy device of claim 9, wherein the outer cannula and inner stylette mechanically imprint an orientation of an acquired tissue sample.

11. The biopsy device of claim 9, comprising at least one Hall effect sensor configured to monitor a stroke of the outer cannula, wherein the stroke is controlled in response to an indication from the at least one Hall effect sensor.

12. The biopsy device of claim 9, wherein a stroke of the outer cannula is controlled in response to a monitored stall current.

13. The biopsy device of claim 9, wherein the syringe is a disposable syringe.

14. A biopsy visualization system, comprising:
a biopsy device, comprising:
a housing configured to receive a syringe therein, the syringe comprising an outer needle and an inner stylette including a sensor in a distal end of the inner stylette; and
a syringe drive assembly configured to engage with a cylinder of the syringe when installed in the housing, wherein the housing secures a plunger of the syringe in a fixed position within the housing and the syringe drive assembly linearly advances the cylinder within the housing at a constant linear speed when activated; and
a visualized tracking and biopsy system configured to track position of the biopsy device based at least in part upon position of the sensor within a patient and generate a 3D visualization including a location of the biopsy device for rendering.

15. The biopsy visualization system of claim 14, wherein the sensor is a magnetic sensor.

16. The biopsy visualization system of claim 15, wherein the outer needle and inner stylette are fabricated from a non-magnetic material.

17. The biopsy visualization system of claim 14, wherein the sensor is coupled to the visualized tracking and biopsy system via a cable extending from the sensor through the inner stylette.

18. The biopsy visualization system of claim 14, wherein stroke position of the syringe is monitored by Hall effect sensors.

19. The biopsy visualization system of claim 14, wherein the visualized tracking and biopsy system is configured to concurrently track a micro-ultrasound (microUS) probe.

20. The biopsy visualization system of claim 19, wherein the 3D visualization includes a location of the microUS probe.

21. The biopsy visualization system of claim 14, wherein the 3D visualization includes guidance for obtaining a sample from a region of interest with the biopsy device.

22. The biopsy visualization system of claim 21, wherein the visualized tracking and biopsy system records position and orientation of the sample within the region of interest.

23. The biopsy visualization system of claim 14, wherein a sample is obtained by the outer needle, the sample encoded with an orientation indication.
